(19) United States
(12) Patent Application Publication
(10) Pub. No.: US 2003/0007973 A1
(43) Pub. Date: Jan. 9, 2003

(54) Methods and compositions for manipulation of the immune response using anti-metallothionein antibody

(76) Inventor: Michael A. Lynes, Willington, CT (US)

Correspondence Address: CANTOR COLBURN, LLP, 55 GRIFFIN ROAD SOUTH, BLOOMFIELD, CT, 06002

(21) Appl. No.: 10/178,909

(22) Filed: Jun. 24, 2002

Related U.S. Application Data
(60) Provisional application No. 60/300,346, filed on Jun. 22, 2001.

(51) Int. Cl. A61K 39/395

(52) U.S. Cl. 424/146.1

(57) ABSTRACT

Metallothionein is a stress response protein responsible for some forms of stress-related immunosuppression. An anti-metallothionein antibody can stimulate the humoral immune response to T-dependent antigens. An anti-metallothionein antibody is particularly useful when administered with an antigen to stimulate the immune response in a subject. An anti-metallothionein antibody can be administered to a subject with a vaccine to immunize the subject.

### CROSS REFERENCE TO RELATED APPLICATIONS

This application claims priority to Provisional Application Ser. No. 60/300,346, filed Jun. 22, 2001, which is incorporated herein by reference in its entirety.

### STATEMENT REGARDING FEDERALLY SPONSORED RESEARCH

The U.S. Government has certain rights in this invention pursuant to Grant No. NIEHS ES07408 awarded by NIH.

### TECHNICAL FIELD

This disclosure relates to the regulation of the humoral immune response to T-dependent antigens.

### BACKGROUND

The induction of an immune response depends on many factors, among which are the chemical composition and configuration of the immunogen, the immunogenic constitution of the challenged organism, and the manner and period of administration of the immunogen. The immunogen presents the subject with immune-stimulating compounds called antigens, which then provoke one or more different responses from the subject to destroy or eliminate the immunogen. The immune response is conveniently divided into two main categories: humoral and cell-mediated. The humoral component of the immune response is mediated by antibodies, molecules circulating in the blood and lymph that specifically interact with the antigenic determinants of antigens. B lymphocytes are the cells responsible for the production of antibodies. The cell-mediated component of the immune response includes the leukocytes or white blood cells. The T lymphocytes include cells that regulate the vigor of the immune response, and other cells that participate in the killing of target cells. The development and maintenance of an individual's protective immune response to an immunogen depends on the achievement of a critical level of stimulation of both the cell-mediated and humoral immune responses.

Ideally, an immunogen can exhibit two properties: the capacity to stimulate the formation of the corresponding antibodies, and the propensity to react specifically with these antibodies. Immunogens bear one or more epitopes, which are the smallest part of an immunogen recognizable by the combining site of an antibody. An example of an immunogen is a antigen from a pathogenic organism.

The initial presentation of an immunogen or antigen induces the production of both IgM and IgG antibodies, forming the primary immune response. The production of antibodies may, however, take time to develop and diminish with time. A secondary immune response involving the production of IgG antibodies is triggered far more rapidly than the primary immune response upon a second presentation of immunogen or booster.

The immune response can be the result of the initial or priming dose or one or more booster exposures to the immunogen to induce the secondary immune response. Priming with relatively strong immunogens and liposomes is discussed in “Liposomal Enhancement of the Immunogenicity of Adenovirus Type 5 Hexon and Fiber Vaccines”, Kramp, W. J. et al., Infection and Immunity 25:771-773 (1979) and “Liposomes as Adjuvants with Immunopurified Tetanus Toxoid: the Immune Response”, Davis, D. et al., Immunology Letters 14:341-8 (1986/1987). A secondary or even a primary immune response is not certain simply by priming the subject with an antigen.

A difficulty often encountered with the administration of an antigen is the extent of the response of the immune system. Certain immunogens elicit a poor primary immune response, or no response at all. This is often the case with fractions of immunogens, peptides, or other small molecules used as immunogens. In these cases, the immune system may not respond to a secondary challenge with immunogen. In extreme cases, the subject may suffer from the condition that immunization was supposed to prevent.

The vaccine art recognizes the use of certain substances called adjuvants that can potentiate an immune response when used in conjunction with an immunogen. Adjuvants are used to elicit an immune response that is faster or greater than would be elicited without the use of the adjuvant. In addition, adjuvants may be used to create an immunological response using less immunogen than would be needed without the inclusion of adjuvant, to increase production of certain antibody subclasses that afford immunological protection or to enhance components of the immune response (e.g., humoral, cellular). Known adjuvants include Freund's Adjuvants (and other oil emulsions), Bordetella Pertussis, aluminum salts (and other metal salts), and Mycobacterial products (including muramyl dipeptides). More recent versions of adjuvants include interleukins (U.S. Pat. Nos. 6,168,923 and 5,747,024; and Arulanaandam et al., J. Immunol 164:3698-704 (2000)), Cholera toxin (Scharton-Kersten et al., Vaccine 17: Suppl 2:S37-43 (1999)), liposomes (U.S. Pat. Nos. 4,053,585 and 6,090,406), peptides (U.S. Pat. No. 6,100,380) and nucleic acids (U.S. Pat. No. 6,239,116; and Gallichan et al., J. Immunol. 155:3451-7 (2001)). Adjuvants may be in a number of forms including emulsions (e.g., Freund's adjuvant), gels (e.g., aluminum hydroxide gel), particles (e.g., liposomes) or solid materials.

With many adjuvants, adverse reactions are seen. In certain cases, adverse reactions may include granuloma formation at the site of injection, severe inflammation at the site of injection, pyrogenicity, adjuvant induced arthritis or other autoimmune response, or oncogenic response. Such reactions have hampered the use of adjuvants such as Freund's adjuvant. Because of the negative effects seen with many adjuvants, human vaccines rarely contain adjuvants.

Accordingly, there remains a need for compositions such as adjuvants that can increase the response of the immune system without producing negative responses in the subject. Such compositions may be suitable for use in pharmaceutical compositions such as vaccines.

### BRIEF SUMMARY

The above-discussed and other drawbacks and deficiencies of the prior art are alleviated by a method for treating a subject comprising administering to the subject a composition comprising an amount of an anti-metallothionein antibody effective to stimulate a humoral immune response in the subject.

Also disclosed herein is a method of treating a subject comprising administering to the subject an antigen, and administering to the subject a first amount of an anti-metallothionein antibody effective to stimulate a humoral immune response in the subject.

Further disclosed is a method of immunizing a subject comprising administering to the subject a composition comprising a vaccine and an anti-metallothionein antibody.

A method of staining a cell comprising contacting the cell with an anti-metallothionein antibody and detecting the metallothionein-anti-metallothionein antibody complexes formed is also disclosed.

A method of recovering metallothionein from a mixture of proteins comprising contacting the mixture of proteins with an anti-metallothionein antibody and isolating the metallothionein-anti-metallothionein antibody complexes formed is disclosed herein.

A method of screening for compounds useful for stimulating the humoral immune response comprising contacting a test compound with metallothionein, and detecting the presence or absence of binding to metallothionein is disclosed. The presence of binding to metallothionein indicates that the test compound potentially increases the humoral immune response.

Further disclosed is a method to assess the stimulatory activity of a test substance on the humoral immune response, comprising contacting metallothionein with the test substance, detecting the humoral immune response in the presence of the test substance, and comparing the humoral immune response in the presence of the test substance with the humoral response in the absence of the test substance.

The above-discussed and other features and advantages of the present disclosure will be appreciated and understood by those skilled in the art from the following detailed description and drawings.

### BRIEF DESCRIPTION OF THE DRAWINGS

Referring now to the exemplary drawings wherein like elements are numbered alike in the several FIGURES:

FIG. 1 shows the anti-OVA (ovalbumin) IgG response in BALB/cJ mice co-injected with an anti-metallothionein (metallothionein) antibody (UC1MT) or isotype control (IgG₁-kappa). The points represent the OVA injected control group (▪), the group injected with OVA and UC1MT (♦), and the group injected with OVA and an Ig control (●). Groups of five female BALB/c mice were injected intraperitoneally with 100 μg OVA alone or OVA combined with 100 μg UC1MT or an isotype control at Day 0 and Day 10. Samples of peripheral blood were drawn starting at day 14 after the initial immunization. Anti-OVA IgG responses were then determined by ELISA for the time points indicated. Results are graphed as the average for each group plus or minus the standard deviation for that group.

FIG. 2 shows the anti-OVA IgG1 responses in BALB/c mice co-injected with an anti-metallothionein antibody (UC1MT) or isotype control (IgG1-kappa). The points represent the OVA injected control group (▪), the group injected with OVA and UC1MT (♦), and the group injected with OVA and an Ig control (●). Anti-OVA IgG1 responses were determined by ELISA at the times indicated.

FIG. 3 shows the anti-OVA IgG2a responses in BALB/c mice co-injected with an anti-metallothionein antibody (UC1MT) or isotype control (IgG1-kappa). The points represent the OVA injected control group (▪), the group injected with OVA and UC1MT (♦), and the group injected with OVA and an Ig control (●). Anti-OVA IgG1 responses were determined by ELISA at the times indicated. The results are plotted on the same scale as the IgG1 responses shown in FIG. 2 and in an expanded scale in the inset.

FIG. 4 shows an ELISPOT assay specific for anti-OVA IgG in mice injected with OVA alone, OVA+UC1MT and OVA+Ig Control. The enzyme-linked immunospot (ELISPOT) assay was performed on day 15 following the second injection at Day 10. Splenocytes from each group of mice were incubated overnight in a 96-well filtration plate. Individual spots were counted in coded wells.

FIG. 5 shows the cytometric analysis of splenocytes from immunized mice for the expression of cell surface markers. Splenocytes (1×10⁶ cells/well) were stained with anti-mouse IgG+IgM-FITC (panels A-C), anti-mouse CD4-FITC (panels D-F) and anti-mouse CD8-APC (panels G-I). Mice immunized with OVA+UC1MT (panels B, E, H) did not differ in their expression of cell surface markers when compared to the control groups (panels A, D, G) and (panels C, F, I). Numbers over the marker region refer to the percent of total cells in that region.

FIG. 6 shows binding of UC1MT-FITC (fluorescein isothiocyanate). Binding to splenocytes after 15 hours in culture with or without metallothionein. Splenocytes were incubated in the presence and absence of metallothionein (20 μM) for 15 hours. Following incubation, splenocytes were stained with UC1MT-FITC or MOPC 21-FITC. Cells were incubated with MOPC 21-FITC in the presence or absence of exogenous metallothionein were essentially equivalent to the unlabeled control in the fluorescence intensity of the cells. The lines are: UC1MT-FITC without metallothionein ( [symbol] ), UC1MT-FTIC with metallothionein (&Rectsolid;), MOPC 21-FTIC without metallothionein (---), MOPC 21-FTIC with metallothionein ( . . . ), and a blank (---).

FIG. 7 shows binding of conjugated antibodies to metallothionein on splenocytes after 40 hours. Splenocytes (1×10⁶ cells/well) from BALB/cJ mice were incubated in the presence and absence of metallothionein (20 μM/well) for 40 hours and then, stained with UC1MT-FITC and MOPC 21-FITC. The lines are: UC1MT-FITC without metallothionein ( [symbol] ), UC1MT-FTIC with metallothionein (&Rectsolid;), MOPC 21-FTIC without metallothionein (---), MOPC 21-FTIC with metallothionein ( . . . ), and a blank (---).

FIG. 8 shows binding of FITC conjugated antibodies to metallothionein on splenocytes from mev/mev autoimmune mice and +/mev mice. Splenocytes from C57BL/6J-Hcph^{mev} (abbreviated as mev/mev) and +/mev mice were incubated with UC1MT-FITC without incubating with exogenous metallothionein. Although splenocytes from mev/mev homozygotes were more highly stained with UC1MT-FITC, splenocytes from littermate controls (+/mev) remained unstained. The lines are: mev/mev (UC1MT-FITC) ( [symbol] ), +/mev (UC1MT-FTIC) (&Rectsolid;), mev/mev unstained ( [symbol] ), and +/mev unstained ( . . . ). These results are representative of two independent experiments.

FIG. 9 shows the amino acid sequence of the foot and mouth disease virus (FMDV) VP1-A12₁₄₁₋₁₅₉ region of two closely related variants (FP and FL). The putative cell attachment domain is contained in the “L” sub-epitope VP1-A12₁₄₁₋₁₅₉, and the putative virus internalization domain is within the “R” sub-epitope VP1-A12₁₄₁₋₁₅₉. The FL variant differs only at residue 153 from wild-type FP.

FIG. 10 shows the FACS analysis of extracellular UC1MT binding in guinea pig splenocytes.

FIG. 11 shows FACS analysis of intracellular UC1MT binding in blood leukocytes from immunized and unimmunized animals. Cells from FMDV+RIBI immunized animals are most intensively stained with UC1MT when compared to unimmunized and FMDV+PBS immunized animals.

FIG. 12 shows the guinea pig anti-FMDV specific IgG response in guinea pigs co-injected with anti-metallothionein antibody or an isotype control (MOPC 21). Serum samples collected from immunized animals were tested by ELISA at the times indicated. The results show the anti-metallothionein antibody does not enhance the anti-FMDV response. FIG. 13 shows the guinea pig anti-UC1MT response in animals co-injected with UC1MT or MOPC 21 in the presence of RIBI adjuvant. Anti-UC1MT response was determined by ELISA at day 14. The results show that FMDV+RIBI immunized animals had increased levels of anti-UC1MT response.

FIG. 13 shows the guinea pig anti-UC1MT response in animals co-injected with UC1MT or MOPC 21in the presence of RIBI adjuvant. Anti-UC1MT response was determined by ELISA at day 14. The results show that FMDV+RIBI immunized animals had increased levels of anti-UC1MT response.

FIG. 14 shows guinea pig anti-MOPC 21 response in animals co-injected with UC1MT of MOPC 21 in the presence of RIBI adjuvant. Anti-MOPC 21 response was determined by ELISA at day 14. The UC1MT injected group had increased levels of anti-MOPC 21 response when compared to the MOPC 21 injected group.

### DETAILED DESCRIPTION

Biological stress can be responsible for suppression of antigen-specific immune responses. This is true for a broad range of stressors, including a subject of different environmental toxicants, psychological stressors, certain infections, and inflammation. Vaccination can also elicit stress, both in the sense that the act of vaccination causes tissue wounding and psychological stress, and in the sense that vaccination results in stress arising from increased metabolic activity of the activated immune cells. It is shown herein that a stress response protein, metallothionein, is responsible for some forms of stress-related immunosuppression. Moreover, it is shown that an anti-metallothionein antibody can inhibit metallothionein-related immunosuppression and can significantly augment the humoral immune response to a prototype T-dependent antigen (ovalbumin). The present disclosure thus relates to manipulations of metallothionein using an anti-metallothionein antibody to enhance both the humoral and cellular immune response. Increasing the immune response is useful in vaccine applications, including agriculturally important vaccines.

Metallothionein (metallothionein) is stress response protein that binds heavy metals with high affinity (Hamer, Ann. Rev. Biochem. 55:913-51 (1986)). The synthesis of metallothionein in the cell is stimulated by the addition of heavy metals, free radicals, irradiation, acute phase cytokines such as interleukin-1 (IL-1), interleukin-6 (IL-6) and tumor necrosis factor-α (TNF-α), inflammatory agents such as lipopolysaccharide (LPS), and alkylating agents. Metallothionein is predominantly synthesized in liver (Coyle et al. Inflamm. Res 44:475-81 (1995); Quaife et al. Biochemistry 33:7250-9 (1999)), but other cells and tissues including lymphocytes, monocytes, and lymphoid tissues like the thymus can also produce metallothionein under the appropriate stimuli (Coto et al. Proc. Nat. Acad. Sci, U.S.A. 89:7752-6 (1992)). Mammals express four major isoforms of metallothionein. Metallothionein-I and metallothionein-II are the primary inducible isoforms of metallothionein, and can be expressed in most vertebrate tissues (Hamer, Ann. Rev. Biochem. 55:913-51 (1986)). Mice deficient in the expression of metallothionein-I and metallothionein-II have been produced by targeted disruption of the MT1 and MT2 genes (metallothioneinKO mice). These mice have been used to determine the role of metallothionein in the response to heavy metals (Masters et al., Proc. Nat. Acad. Sci. U.S.A. 91:584-8 (1994)), UV irradiation (Michalska and Choo, Proc. Nat. Acad. Sci. U.S.A. 90:8088-92 (1993); Reeve et al. Immunology 100:399-404 (2000)) and to explore the roles of metallothionein in immune function (Crowthers et al., Toxicol. App. Pharm. 166:161-72 (2000)). metallothionein binds a number of metals including cadmium and mercury, so metallothionein has been thought to play a protective role in cells and organisms exposed to heavy metals. metallothionein functions as a potent antioxidant by scavenging free radicals. metallothionein can also serve as a reservoir of essential metals such as zinc and copper, which are required for growth and development.

Beyond these fundamental physiological roles, or perhaps as a consequence of these roles, metallothionein has been shown to have several immunomodulatory effects. metallothionein induces lymphocyte proliferation when added alone to splenocyte cultures, and can also act synergistically with other activators of B and T lymphocytes (e.g. lipopolysaccharide (LPS) and Concanavalin A (Con A) (Lynes et al., Mol. Immunol. 27:2119-9 (1990)) to stimulate cell division. metallothionein can also decrease the in vivo humoral immune responses to T-dependent antigens. Injection of exogenous metallothionein suppresses the specific anti-ovalbumin (OVA) response.

The cellular function of metallothionein has been further elucidated by the use of a monoclonal antibody raised against metallothionein, the antibody UC1Mt. Co-injected UC1MT (a monoclonal anti-metallothionein antibody) blocks exogenous metallothionein-mediated suppression of the anti-OVA response (Lynes et al. Toxicology 85:161-77 (1993)). Comparison of humoral immune function in mice with a targeted knockout of the MT-1 and 2 genes (metallothioneinKO) with wild-type mice challenged with ovalbumin (OVA) demonstrates that endogenous metallothionein can modulate the immune response. The metallothioneinKO mice have an increased immune response over the wild-type mice suggesting that the absence of metallothionein can serve to increase immune function.

The present disclosure is based in part on the unexpected discovery that in vivo injection of an anti-metallothionein antibody significantly increases the humoral immune response to simultaneous challenge with ovalbumin. As used herein, the term “adjuvant” will be understood to broadly mean a substance or material administered together or in conjunction with an immunogen that increases or modifies the immune response to that immunogen.

The present disclosure includes isolated (i.e., removed from their natural milieu) antibodies that selectively bind metallothionein or a mimetope thereof. As used herein, the term “selectively binds to” refers to the ability of antibodies of the present disclosure to preferentially bind to metallothionein and mimetopes thereof. Binding can be measured using a variety of methods standard in the art including enzyme immunoassays (e.g., ELISA), immunoblot assays, and the like; see, for example, Sambrook et al., Eds., Molecular Cloning: A Laboratory Manual, 2nd ed., Cold Spring Harbor Laboratory Press, 1989, or Harlow and Lane, Eds., Using Antibodies, Cold Spring Harbor Laboratory Press, 1999. An anti-metallothionein antibody selectively binds to or complexes with metallothionein, preferably in such a way as to reduce the activity of metallothionein.

As used herein, anti-metallothionein antibody includes antibodies in serum, or antibodies that have been purified to varying degrees, preferably at least about 25%. The anti-metallothionein antibodies are preferably purified to at least about 50% homogeneity, more preferably at least about 75% , and most preferably greater than about 90% homogeneity. Anti-metallothionein antibodies may be polyclonal antibodies, monoclonal antibodies, humanized or chimeric antibodies, anti-idiotypic antibodies, single chain antibodies, Fab fragments, fragments produced from an Fab expression library, epitope-binding fragments of the above, and the like. An anti-metallothionein antibody can be a biologically active fragment, that is, a fragment of a full-length anti-metallothionein antibody that is capable of binding metallothionein. Biologically active fragments include Fab, F(ab′)₂ and Fab′ fragments. Preferred anti-metallothionein antibodies are about 25 kDa (kilodaltons) to about 900 kDa, preferably about 50 kDa to about 300 kDa in size.

Antibodies that bind to metallothionein are prepared by immunizing an animal with full-length metallothionein polypeptide or fragments of the metallothionein polypeptide. Preferred fragments of the metallothionein polypeptide are those containing metallothionein-specific epitopes. The preparation of polyclonal antibodies is well known in the molecular biology art; see for example, Production of Polyclonal Antisera in Immunochemical Processes (Manson, ed.), (Humana Press 1992) and Coligan et al., Production of Polyclonal Antisera in Rabbits, Rats, Mice and Hamsters in Current Protocols in Immunology, (1992).

A monoclonal antibody composition can b produced, e.g., by clones of a single cell called a hybridoma that secretes or otherwise produces one kind of antibody molecule. Hybridoma cells can be formed by fusing an antibody-producing cell and a myeloma cell or other self-perpetuating cell line. The preparation of monoclonal antibodies was first described by Kohler and Milstein, Nature 256:495-497 (1975), although numerous variations have been described for producing hybridoma cells.

Briefly, monoclonal antibodies can be obtained by injecting mammals such as mice or rabbits with a composition comprising an antigen, thereby inducing in the animal antibodies having specificity for the antigen. A suspension of antibody-producing cells is then prepared (e.g., by removing the spleen and separating individual spleen cells by methods known in the art). The antibody-producing cells are treated with a transforming agent capable of producing a transformed or “immortalized” cell line. Transforming agents are known in the art and include such agents as DNA viruses (e.g., Epstein Bar Virus, SV40), RNA viruses (e.g., Moloney Murine Leukemia Virus, Rous Sarcoma Virus), myeloma cells (e.g., P3X63-Ag8.653, Sp2/0-Ag14) and the like. Treatment with the transforming agent can result in production of a hybridoma by means of fusing the suspended spleen cells with, for example, mouse myeloma cells. The transformed cells are then cloned, preferably to monoclonality. The cloning is preferably performed in a medium that will not support non-transformed cells, but that will support transformed cells. The tissue culture medium of the cloned hybridoma is then assayed to detect the presence of secreted antibody molecules by antibody screening methods known in the art. The desired clonal cell lines are then selected.

A therapeutically useful anti-metallothionein antibody may be derived from a “humanized” monoclonal antibody. Humanized monoclonal antibodies are produced by transferring mouse complementarity determining regions from heavy and light variable chains of the mouse immunoglobulin into a human variable domain, then substituting human residues into the framework regions of the murine counterparts. The use of antibody components derived from humanized monoclonal antibodies obviates potential problems associated with immunogenicity of murine constant regions. Techniques for producing humanized monoclonal antibodies can be found in Jones et al., Nature 321: 522 (1986) and Singer et al., J. Immunol. 150: 2844 (1993). The antibodies can also be derived from human antibody fragments isolated from a combinatorial immunoglobulin library; see, for example, Barbas et al., Methods: A Companion to Methods in Enzymology 2, 119 (1991).

In addition, chimeric antibodies can be obtained by splicing the genes from a mouse antibody molecule with appropriate antigen specificity together with genes from a human antibody molecule of appropriate biological specificity; see, for example, Takeda et al., Nature 314: 544-546 (1985). A chimeric antibody is one in which different portions are derived from different animal species.

Anti-idiotype technology can be used to produce monoclonal antibodies that mimic an epitope. An anti-idiotypic monoclonal antibody made to a first monoclonal antibody will have a binding domain in the hypervariable region that is the “image” of the epitope bound by the first monoclonal antibody. Alternatively, techniques used to produce single chain antibodies can be used to produce single chain antibodies against metallothionein, as described, for example, in U.S. Pat. No. 4,946,778. Single chain antibodies are formed by linking the heavy and light chain fragments of the Fv region via an amino acid bridge, resulting in a single chain polypeptide.

Antibody fragments that recognize specific epitopes can be generated by techniques well known in the art. Such fragments include Fab and F(ab′)₂ fragments produced by proteolytic digestion, and Fab′ fragments generated by reducing disulfide bridges. Fab, F(ab′)₂ and Fab′ fragments of anti-metallothionein antibodies can be prepared. Fab fragments are typically about 50 kDa, while F(ab′)₂ fragments are typically about 100 kDa in size. Anti-metallothionein antibodies can be isolated (e.g., on protein G columns) and then digested and purified with sepharose coupled to papain and to pepsin in order to purify Fab and F(ab′)₂ fragments according to protocols provided by the manufacturer (Pierce Chemical Co.). The antibody fragments can be further purified, isolated and tested using ELISA assays. Antibody fragments can be assessed for the presence of light chain and Fc epitopes by ELISA.

In another method, anti-metallothionein antibodies of the present disclosure can be produced recombinantly using techniques known in the art. Recombinant DNA methods for producing antibodies include isolating, manipulating, and expressing the nucleic acid that codes for all or part of an immunoglobulin variable region including both the portion of the variable region comprised by the variable region of the immunoglobulin light chain and the portion of the variable region comprised by the variable region of the immunoglobulin heavy chain. Methods for isolating, manipulating and expressing the variable region coding nucleic acid in eukaryotic and prokaryotic subjects are disclosed in U.S. Pat. No. 4,714,681; Sorge et al., Mol. Cell. Biol. 4:1730-1737 (1984); Beher et al., Science 240:1041-1043 (1988); Skerra et al., Science 240:1030-1041 (1988); and Orlandi et al., Proc. Natl. Acad. Sci. U.S.A. 86:3833-3837 (1989).

The structure of the anti-metallothionein antibody may also be altered by changing the biochemical characteristics of the constant regions of the antibody molecule to a form that is appropriate to the particular context of the antibody use. For example, the isotype of the antibody may be changed to an IgA form to make it compatible with oral administration. IgM, IgG, IgD, or IgE isoforms may have alternate values in the specific therapy in which the antibody is used.

Antibodies raised against metallothionein or mimetopes can be advantageous because such antibodies are not substantially contaminated with antibodies against other substances that might otherwise cause interference in a diagnostic assay or side effects if used in a therapeutic composition.

A preferred method to produce anti-metallothionein antibodies includes (a) administering to an animal or a collection of cultured competent immune cells from human or animal, an effective amount of metallothionein, a fragment thereof, or a mimetope thereof, to produce the antibodies and (b) recovering the antibodies. Antibodies can be purified by methods known in the art. Suitable methods for antibody purification include purification on Protein A or Protein G beads, protein chromatography methods (e.g., DEAE ion exchange chromatography, ammonium sulfate precipitation), antigen affinity chromatography and others.

Anti-metallothionein antibodies have a variety of uses within the scope of the present disclosure. In one embodiment, anti-metallothionein antibodies are used to stain cells to detect the presence of metallothionein, by contacting the metallothionein antibody with the cells and detecting the metallothionein-anti-metallothionein antibody complex formed. The metallothionein antibody may be labeled. Anti-metallothionein antibodies can also be used determine the localization of metallothionein in its physiological setting by labeling the anti-metallothionein antibody; contacting the labeled anti-metallothionein antibody with the tissues or cells of interest; and detecting the labeled metallothionein-anti-metallothionein antibody complex formed. Labeling may be by means of fluorescent tags, radioactivity, or other methods known in the art.

An anti-metallothionein antibody may further be used to purify or isolate metallothionein from a mixture, in particular a mixture of proteins and/or other contaminants. The method comprises contacting the mixture with an anti-metallothionein antibody; and isolating metallothionein-anti-metallothionein antibody complexes formed. The antibody may, for example be immobilized on a column to facilitate separation of the metallothionein from the mixture.

One embodiment herein comprises methods for the identification of metallothionein binding molecules that potentially have therapeutic activity by affecting the immunosuppressive activity of metallothionein. In particular, a method of screening for compounds useful for stimulating the humoral immune response comprises contacting a test compound with metallothionein, and detecting the presence or absence of binding to metallothionein. The presence of binding to metallothionein indicates that the test compound potentially increases the humoral immune response. Such metallothionein binding molecules can increase the humoral immune response to T-dependent antigens.

Test compounds can encompass many classes such as organic molecules, polypeptides and nucleic acids. Organic molecules and polypeptides having effective activity may be designed de novo, identified through assays or screens of large libraries, e.g., or obtained by a combination of the two techniques. Non-protein drug design may be carried out using computer graphic modeling to design non-polypeptide, organic molecules able to bind metallothionein. Techniques for constructing and screening combinatorial libraries of small molecules or oligomeric biomolecules to identify those that specifically bind to a given receptor protein are known. As used herein, “combinatorial library” refers to collections of diverse oligomeric biomolecules of differing sequence, which can be screened simultaneously for activity as a ligand for a particular target. Combinatorial libraries may also be referred to as “shape libraries”, i.e., a population of randomized fragments that are potential ligands. The shape of a molecule refers to those features of a molecule that govern its interactions with other molecules, including Van der Waals, hydrophobic, electrostatic and dynamic.

Libraries may be synthesized in solution on solid supports, or expressed on the surface of bacteriophage viruses (phage display libraries). Libraries of compounds may be presented in solution (e.g., Houghten, Biotechniques 13: 412-421, 1992), or on beads (Lam, Nature 354: 82-84 (1991)), chips (Fodor, Nature 364: 555-556 (1993)), bacteria spores, plasmids, or phage (Ladner, U.S. Pat. No. 5,223,409).

Known screening methods may be used by those skilled in the art to screen combinatorial libraries to identify active metallothionein binding molecules. Screening methods can be carried out in a cell or cells, or can be carried out in essentially cell free preparations. The method may be carried out as a single assay, or may be implemented in the form of a high throughput screen in accordance with a variety of known techniques. The method of screening test compounds can comprises determining in vitro whether the test compound specifically binds to metallothionein. Alternatively, binding can be determined by an increase (or decrease) in the interaction of metallothionein with proteins at the immune synapse can be used to screen compounds. Other techniques are known in the art for screening synthesized molecules to select those with the desired activity, and for labeling the members of the library so that selected active molecules may be identified, as in U.S. Pat. No. 5,283,173 to Fields et al. (use of genetically altered Saccharomyces cerevisiae to screen peptides for interactions).

Candidate metallothionein binding molecules encompass many chemical classes. They can be organic molecules, preferably organic compounds having molecular weights of 50 to 2,500 daltons. The candidate molecules comprise functional groups necessary for structural interaction with proteins, particularly hydrogen bonding, for example, carbonyl, hydroxyl, and carboxyl groups. The candidate molecules can comprise cyclic carbon or heterocyclic structures and aromatic or polyaromatic structures substituted with the above groups.

Suitable oligomers include polypeptides, oligonucleotides, carbohydrates, oligonucleotides analogs (e.g., phosphorothioate oligonucleotides; see Chem. and Engineering News, page 20, Feb. 7, 1994) and nonpeptide polymers (see, e.g., “peptoids” of Simon et al., Proc. Natl. Acad. Sci. USA 89:9367 (1992)). See also U.S. Pat. No. 5,270,170 to Schatz; Scott and Smith, Science 249: 386-390 (1990); Devlin et al., Science 249: 404-406 (1990); Edgington, BIO/Technology 11: 285 (1993).

Metallothionein binding molecules may also be nucleic acid molecules (Edgington, BIO/Technology 11:285 (1993)). U.S. Pat. No. 5,270,163 to Gold and Tuerk describes a method for identifying nucleic acid ligands for a given target molecule by selecting from a library of RNA molecules with randomized sequences those molecules that bind specifically to the target molecule. A method for the in vitro selection of RNA molecules immunologically cross-reactive with a specific polypeptide is disclosed in Tsai, Kenan and Keene, Proc. Natl. Acad. Sci. USA 89: 8864 (1992); and Tsai and Keene, J. Immunology 150: 1137 (1993). In the method, an antiserum raised against a polypeptide is used to select RNA molecules from a library of RNA molecules; selected RNA molecules and the polypeptide compete for antibody binding, indicating that the RNA epitope functions as a specific inhibitor of the antibody-antigen interaction.

Macromolecules that interact with metallothionein are referred to as metallothionein binding partners. Metallothionein binding partners are likely to be involved in the regulation of metallothionein function. Therefore, it is possible to identify compounds that interfere with the interaction between metallothionein and its binding partners. The present disclosure also includes a method for identifying molecules that affect the binding of metallothionein and a metallothionein binding partner. A reaction mixture containing metallothionein or an metallothionein fragment and the binding partner under conditions that allow complex formation is prepared in the presence or absence of the test compound to test for activity. The test compound may be added prior to or subsequent to metallothionein/binding partner complex formation. The formation of a complex in a control, but not with the test compound, confirms that the test compound affects complex formation. The assay can be conducted either in the solid phase or in the liquid phase.

Proteins that are metallothionein binding partners can be detected by methods known in the art. One method to detect protein-protein interactions in vivo is the two-hybrid system, see, for example, Chien et al., Proc. Natl. Acad. Sci, USA 88: 9578-9582 (1991). In brief, the two-hybrid system utilizes plasmids constructed to encode two hybrid proteins: one plasmid comprises the nucleotides encoding the DNA binding domain of a transcriptional activator protein fused to the metallothionein nucleotide sequence encoding the metallothionein polypeptide, and the other plasmid comprises the nucleotides encoding the transcriptional activator protein's activation domain fused to a cDNA encoding an unknown protein that has been recombined into the plasmid from a cDNA library. The DNA binding domain fusion plasmid and the cDNA fusion protein library plasmids are transformed into a strain of yeast that contains a reporter gene, for example lacZ, whose regulatory region contains the activator's binding site. Either hybrid protein alone cannot activate translation of the reporter gene because it is lacking either the DNA binding domain or the activator domain. Interaction of the two hybrid proteins, however, reconstitutes a functional activator protein and results in activation of the reporter gene that is detected by an assay for the reporter gene product. The colonies that reconstitute activator activity are purified and the library plasmids responsible for reporter gene activity are isolated and sequenced. The DNA sequence is then used to identify the protein encoded by the library plasmid.

Metallothionein binding molecules and molecules that interfere with metallothionein/metallothionein binding partner interactions are also potentially useful to modulate the humoral immune response. A method to assess the stimulatory activity of a test substance on the humoral immune response comprises contacting metallothionein with the test substance, detecting the humoral immune response in the presence of the test substance, and comparing the humoral immune response in the presence of the test substance with the humoral response in the absence of the test substance. Stimulation of the humoral immune response in the presence as compared to the absence of the test substance indicates that the test substance is an immune function stimulator.

The humoral immune response may be measured by many well-known methods. Single Radial Immunodiffusion Assay (SRID), Enzyme lnnunoassay (EIA), and Hemagglutination Inhibition Assay (HAT) are but a few of the commonly used assays of humoral immune response. SRID utilizes a layer of a gel such as agarose containing the immunogen being tested. A well is cut in the gel and the serum being tested is placed in the well. Diffusion of the antibody out into the gel leads to the formation of a precipitation ring whose area is proportional to the concentration of the antibody in the serum being tested. EIA, also known as ELISA (Enzyme Linked Immunoassay), is used to determine total antibodies in a sample. The immunogen is adsorbed to the surface of a microtiter plate. The test serum is exposed to the plate followed by an enzyme-linked immunoglobulin, such as IgG. The enzyme activity adherent to the plate is quantified by any convenient means such as spectrophotometry and is proportional to the concentration of antibody directed against the immunogen present in the test sample. HAI utilizes the capability of an immunogen such as viral proteins to agglutinate chicken red blood cells (or the like). The assay detects neutralizing antibodies, i.e., those antibodies able to inhibit hemagglutination. Dilutions of the test serum are incubated with a standard concentration of immunogen, followed by the addition of the red blood cells. The presence of neutralizing antibodies will inhibit the agglutination of the red blood cells by the immunogen.

Anti-metallothionein antibodies can be conjugated (i.e., stably joined) to various agents using techniques known to those skilled in the art. As mentioned above, conjugation with fluorescent agents, radioisotopes, and the like can aid in detection or isolation of metallothionein. In another embodiment, anti-metallothionein antibodies can be used as agents that will bind with metallothionein and by binding, alter (preferably reduce) the activity of metallothionein. In certain cases, the anti-metallothionein antibody can be further conjugated with agents that enhance the activity of the antibody. It is also contemplated that the anti-metallothionein antibodies can be conjugated to moieties that themselves alter (preferably reduce) the activity of metallothionein. In such cases, the anti-metallothionein antibodies act as carriers for the active moiety.

It is expected that reduction of the activity of metallothionein by any of the above methods will enhance the immune response, particularly the humoral immune response. Embodiments of the present disclosure thus include therapeutic methods for treating a subject comprising administering an anti-metallothionein antibody. Such methods include use of an anti-metallothionein antibody as an adjuvant for vaccines. Other uses include modulating the immune response of various subjects. Suitable subjects include, but are not limited to, subjects undergoing irradiation or chemotherapy, autoimmune subjects, subjects exposed to immunosuppressive agents, neonates, and subjects having undergone organ or other transplantation. A subject is preferably a vertebrate animal, for example a human, dog, cat, horse, sheep, pig, goat, chicken, monkey, rat, mouse, rabbit or guinea pig.

In another aspect, an anti-metallothionein antibody or biologically active fragment thereof is useful to modify vaccine efficacy. The present disclosure accordingly provides a pharmaceutical composition useful as a vaccine, comprising an antigen from a pathogen and an effective adjuvanting amount of an anti-metallothionein antibody, the resulting composition being capable of eliciting the vaccinated subject's immunity for a protective response to the pathogen. Preferably the anti-metallothionein antibody is generated as described herein, is a biologically active fragment thereof, or is a version of an anti-metallothionein antibody adapted to the immune system of the subject using recombinant DNA techniques known to those skilled in the arts.

As used herein, “vaccine” means a material that can induce immunity in a subject. Immunity is a state of resistance of an individual to an infecting organism or substance. A vaccine can be an organism or material derived from a pathogenic organism that contains an antigen in a noninfectious or innocuous form. The vaccine may be recombinant or non-recombinant. Vaccines can be viral or bacterial in origin. When inoculated in a non-immune subject, the vaccine will produce active immunity to the antigen without causing the disease. Vaccines may take several forms such as a toxoid, a toxin which has been detoxified but retains its major immunological determinants; killed organisms such as typhoid or cholera; attenuated organisms that are the live, but non-virulent forms of pathogens; an antigen encoded by an attenuated organism; a live tumor cell; or an antigen present on a tumor cell.

An anti-metallothionein antibody may be administered simultaneously with the antigen. In another embodiment, an anti-metallothionein antibody is administered sequentially after the antigen. In yet another embodiment, an anti-metallothionein antibody is administered first simultaneously and then subsequent to the antigen. Effective adjuvanting amounts of an anti-metallothionein antibody can be determined by one of skill in the art depending on factors such as the particular vaccine, the subject, the desired effect, the formulation used, and the like.

An anti-metallothionein antibody may be utilized as an adjuvant either alone or in combination with other adjuvants. Other possible adjuvants include liposomes (U.S. Pat. Nos. 4,053,585 and 6,090,406), cytokines such as interleukin-15 and interleukin-12 (U.S. Pat. Nos. 5,747,024 and 6,168,923), immunostimulatory nucleic acid molecules (U.S. Pat. No. 6,239,116), immunoadjuvant oils (e.g., mineral oil, squalene), polycationic polyelectrolytes (e.g., DEAE-dextran, polyethyleneimine) (U.S. Pat. No. 5,109,026) and mixtures comprising one or more of the foregoing adjuvants.

In a preferred embodiment, an anti-metallothionein antibody can be used as an adjuvant for Foot and Mouth Disease (FMD) vaccines. FMD is a highly infectious viral disease affecting many animals of agricultural importance such as cattle, swine and sheep. It is caused by several strains of virus that belong to the genus Aphthovirus within the family Picomaviridae. Although not fatal, FMD reduces weight and milk output in affected animals. FMD vaccines are killed virus preparations or FMD polypeptides that simulate immunity against particular strains of FMD.

Commercially produced FMD vaccines are typically inactivated virus vaccines. Such inactivated virus vaccines can induce peak antibody titers within 7-21 days, after which the titers begin a gradual decline until the next boost. In many cases, the extremely high titers achieved after immunization confer protection for months or even years, yet a small percentage of animals may become susceptible to infection long before the next scheduled boost. These observations have led some investigators to conclude that inactivated vaccines are unlikely to represent a viable approach to FMDV control. Without being held to theory, it is hypothesized that metallothionein synthesized during vaccination serves to suppress the immune response to the vaccine. A corollary to this hypothesis is that individual animals with the greatest propensity to synthesize metallothionein will be the least responsive to vaccination. Thus, the use of an anti-metallothionein antibody as an adjuvant in FMD vaccine preparations will lead to a decrease in the available metallothionein concentration and thus also to a decrease in the immunosupression caused by metallothionein.

In another aspect, the present disclosure provides a composition comprising an anti-metallothionein antibody in an amount effective to enhance or otherwise modifying immune function following irradiation, chemotherapy treatments or a combination of the foregoing treatments. Cancer and cancer treatments can weaken the immune system, for example by affecting the red blood cells and white blood cells, which help to protect individuals from disease and foreign substances (e.g., bacteria). The present disclosure further relates to a method for enhancing or otherwise modifying immune function following irradiation or chemotherapy treatments, comprising administration of a therapeutically effective amount of a pharmaceutically acceptable composition comprising an anti-metallothionein antibody.

In still another aspect, a composition useful for enhancing or otherwise modifying immune function of autoimmune individuals comprises an amount of an anti-metallothionein antibody effective to therapeutically modify immune function. Autoimmune disease is caused when an individual produces an immune response against the individual's own tissues. Autoimmune diseases include such diseases as Graves disease, rheumatoid arthritis, systemic lupus erythematosis, Type 1 diabetes, pernicious anemia, multiple sclerosis and Sjogrens syndrome. The prevelance of autoimmune disease in the United States is discussed in Jacobson et al., Clin. Immunol. And Immunopath. 84:223-243 (1997). A method for enhancing or otherwise modifying immune function of autoimmune individuals comprises administration of therapeutically effective amount of a pharmaceutically effective composition comprising an anti-metallothionein antibody.

In still another aspect, the present disclosure provides a composition useful for enhancing or otherwise modifying immune function of a subject exposed to an immunosuppressive agent, comprising an amount of an anti-metallothionein antibody effective to therapeutically enhance the immune function of a subject exposed to an immunosuppressive agent, preferably one that stimulates production or activity of metallothionein. Immunosuppressive agents include environmental toxicants and other immunosuppressive agents such as such as tobacco, pesticides, PCBs, and dioxins. A method for enhancing or otherwise modifying immune function of a subject exposed to an immunosuppressive agent comprises administration of a therapeutically effective amount of a pharmaceutically acceptable composition comprising an anti-metallothionein antibody.

In still another aspect, a composition useful for enhancing or otherwise modifying immune function of a neonate comprises an anti-metallothionein antibody. The immune system of the infant is functionally immature. Neonates do not produce much immunoglobulin, and maternal antibodies transferred late in gestation to the fetus offer only short term protection for the neonate. The neonate's immune response to challenge from infectious agents is also deficient. Thus there is a need for agents that will enhance the immune function of neonates. A method for enhancing or otherwise modifying immune function of a neonate comprises administration of a therapeutically effective amount of a pharmaceutically acceptable composition comprising anti-metallothionein antibody.

In still another aspect, a pharmaceutical composition useful for modifying immune function of individuals having undergone organ or other transplantation comprises an anti-metallothionein antibody. A method for enhancing or otherwise modifying immune function of individuals having undergone organ or other transplantation comprises administration of a therapeutically effective amount of a pharmaceutically acceptable composition comprising metallothionein antibody.

Methods for the formulation of pharmaceutically acceptable compositions comprising antibodies are generally known. The subject pharmaceutical formulations may comprise one or more non-biologically active compounds, i.e., excipients, such as stabilizers (to promote long term storage), emulsifiers, binding agents, thickening agents, salts, preservatives, and the like, depending on the route of administration. The composition may further comprise diluents, inert substances useful for adjusting the concentration of various components.

Carriers are usually inactive substances that act as a vehicle for inactive substances. Pharmaceutically acceptable carriers are well known to those in the art. Such carriers include, but are not limited to, large, slowly metabolized macromolecules such as proteins, polysaccharides, polylactic acids, polyglycolic acids, polymeric amino acids, amino acid copolymers, and inactive virus particles. Pharmaceutically acceptable salts can also be used in the composition, for example, mineral salts such as hydrochlorides, hydrobromides, phosphates, or sulfates, as well as the salts of organic acids such as acetates, propionates, malonates, or benzoates. The composition can also contain liquids, such as water, saline, glycerol, and ethanol, as well as substances such as wetting agents, emulsifying agents, or pH buffering agents. Liposomes, such as those described in U.S. Pat. No. 5,422,120, WO 95/13796, WO 91/14445, or EP 524,968 B1, can also be used as a carrier for the therapeutic composition.

For oral administration, the antibody composition may be administered with an inert diluent or with an assimilable edible carrier, or incorporated directly with the food of the diet. The formulations may be incorporated with excipients and used in the form of ingestible tablets, buccal tablets, troches, capsules, elixirs, suspension syrups, wafers, and the like. The tablets, troches, pills, capsules and the like may also contain the following: a binder, such as gum tragacanth, acacia, cornstarch, or gelatin; excipients, such as dicalcium phosphate; a disintegrating agent such as corn starch, potato starch, alginic acid and the like; a lubricant such as magnesium stearate; and a sweetening agents, such as sucrose, lactose or saccharin; a flavoring agent such as peppermint, oil of wintergreen, or the like flavoring. When the dosage unit is a capsule, it may contain, in addition to materials of the above type, a liquid carrier. Various other materials may also be present as coatings or to otherwise modify the physical form of the dosage unit. A syrup or elixir may contain sucrose as a sweetening agent, methyl and propylparabens as preservatives, a dye and flavoring such as cherry or orange flavor. Such additional materials should be substantially non-toxic in the amounts employed. Furthermore, the active agents may be incorporated into sustained-release preparations and formulations. Formulations for parenteral administration may include sterile aqueous solutions or dispersions, and sterile powders for the extemporaneous preparation of sterile, injectable solutions or dispersions.

The solutions or dispersions may also contain buffers, diluents, and other suitable additives, and may be designed to promote the cellular uptake of the active agents in the composition, e.g., liposomes. Sterile injectable solutions are prepared by incorporating the active compounds in the required amount in the appropriate solvent with one or more of the various other ingredients described above, followed by sterilization. Dispersions may generally be prepared by incorporating the various sterilized active ingredients into a sterile vehicle that contains the basic dispersion medium and the required other ingredients from those listed above. In the case of sterile powders used to prepare sterile, injectable solutions, the preferred methods of preparation are vacuum-drying and freeze-drying techniques which yield a powder of the active ingredient plus any additional desired ingredient from previously sterile-filtered solutions. Pharmaceutical formulations for topical administration may be especially useful for localized treatment. Formulations for topical treatment include ointments, sprays, gels, suspensions, lotions, creams, and the like. Formulations for topical administration may include known carrier materials such as isopropanol, glycerol, paraffin, stearyl alcohol, polyethylene glycol, and the like.

The pharmaceutically acceptable carrier may also include a known chemical absorption promoter. Examples of absorption promoters are, e.g., dimethylacetamide (U.S. Pat. No. 3,472,931), trichloroethanol or trifluoroethanol (U.S. Pat. No. 3,891,757), certain alcohols and mixtures thereof (British Patent No. 1,001,949), and British Patent No. 1,464,975. Except insofar as any conventional media or agent is incompatible with the therapeutic active ingredients, its use in the therapeutic compositions and preparations is contemplated. Supplementary active ingredients can also be incorporated into the compositions and preparations.

Dosage levels of the order of from about 0.1 mg to about 140 mg per kilogram of body weight per day are useful in the treatment of the above-indicated conditions (about 0.5 mg to about 7 g per human patient per day). The amount of active ingredient that may be combined with the carrier and/or diluent to produce a single dosage form will vary depending upon the subject treated and the particular mode of administration. Dosage unit forms will generally contain between from about 1 mg to about 500 mg of an active ingredient.

Frequency of dosage may also vary depending on the compound used and the particular condition treated. However, for treatment of most disorders, a dosage regimen of four times daily or less is preferred.

It will be understood, however, that the specific dose level for any particular subject will depend upon a variety of factors including the activity of the specific compound employed, the age, body weight, general health, sex, diet, time of administration, route of administration, and rate of excretion, drug combination and the severity of the particular condition being treated.

The invention is further illustrated by the following non-limiting Examples.

### EXAMPLES

### Example 1

### UC1MT (Monoclonal Anti-Metallothionein Antibody) Treatment Enhances Antigen-Specific Humoral Immune Responses

To determine whether endogenous metallothionein synthesized during the course of a normal immune response regulates the vigor of the humoral arm of that response, female BALB/cJ mice were injected with 100 μg of ovalbumin (OVA) in the absence of exogenous metallothionein. Female BALB/cJ mice (age and sex matched) were obtained from Jackson Laboratory, Bar Harbor, Me., or bred from animals obtained from there. C57BL/6J-HCph^{mev} mev/mev and +/mev littermate controls were produced from breeding pairs generously provided by Dr. Leonard Shultz of the Jackson Laboratory. Mice were housed in a room apart from other colonies, and maintained on a 12:12 light/dark cycle with food and water available ad libitum.

Mice were immunized via intra-peritoneal (i.p.) injection with 100 μg chicken egg ovalbumin (OVA) (Sigma). 100 μg purified UC1MT or 100 μg MOPC 21(isotype control antibody) was injected i.p. in treated groups of mice according to schedules described for the individual experiments. All reagents were prepared in sterile 0.85% NaCl. Small samples of peripheral blood were drawn from the peri-orbital sinus starting at day 14 after the initial immunization, and serum was isolated and stored −20° C. until analyzed. Experimental groups consisted of 5 age and sex matched mice.

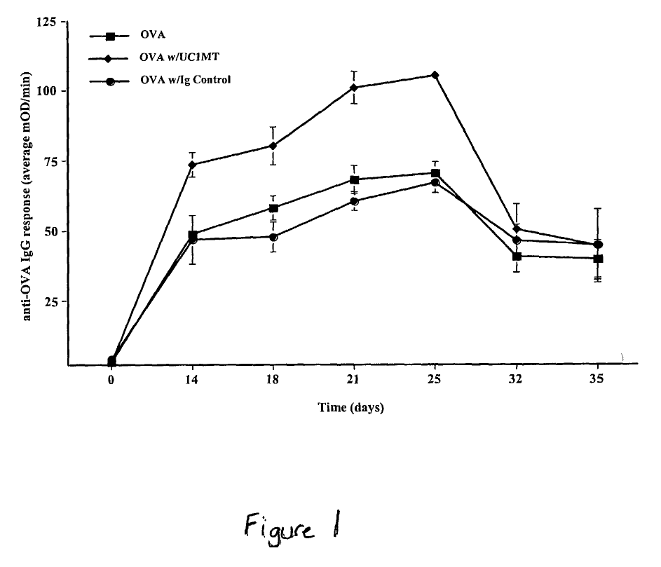

Experimental groups of mice were injected with OVA in combination with UC1MT or with an isotype control (MOPC 21) at day 0 and day 10. The anti-OVA specific antibody response was determined by ELISA at the time points indicated (FIG. 1). Immulon 2 ELISA plates (Dynatech Laboratories, Inc., Alexandria, Va.) were used for these assays. Antigen (OVA) was diluted in coating buffer (1.57% Na₂CO₃, 2.93% NaHCO₃, 0.2% sodium azide, pH 9.7). The plates were incubated with 100 μl antigen/well for 1 hour at 37° C. The plates were aspirated and subsequent non-specific protein absorption was blocked with 200 μl of 1% Teleostean gelatin (Sigma) in phosphate buffered saline (PBS) with Tween 20 and NaN₃. After one hour incubation at 37° C., the plates were washed three times with wash buffer (PBS with 0.2% NaN₃ and 0.005% Tween 20, pH 7.2) in a BioTek EL403 automated plate washer. 100 μl of mouse serum diluted in 1% BSA (1/500 dilution) was then added to individual wells. Following a two hour incubation at 37° C., the plates were washed with wash buffer and incubated for one hour at 37° C. with secondary antibody (alkaline phosphatase conjugates) in 1% BSA. Finally, the wells were washed and 100 μl of substrate (1 mg/ml of para-nitrophenyl phosphate in DEA buffer: 9.7% diethanolamine, 0.02% NaN₃, and 0.01% MgCl₂, pH 9.8) was added to each well. Kinetic color development was determined in a Tmax ELISA microplate reader (Molecular Devices, Menlo Park, Calif.) at 405 nm.

Total immunoglobulin levels were also determined by using ELISA. Briefly, Immulon 2 ELISA plates were coated with 100 μl of capture antibody (goat anti-mouse Ig (H+L), Southern Biotechnology) in coating buffer and incubated overnight at 4° C. The plates were then blocked with 2% BSA in coating buffer for 1 hour at 37° C. After incubation, the plates were washed and 100 82 l of purified immunoglobulin at known concentrations or serum samples from experimental animals diluted in 1% BSA in PBS were added to appropriate wells. The plates were incubated for 1 hour at 37° C., then washed and incubated with goat anti-mouse IgG or IgM specific antibodies conjugated to alkaline phosphatase (Southern Biotechnology). Color development and measurement was performed as described in the previous section.

A significant increase (t test p=0.006) in the anti-OVA IgG response was observed in mice co-injected with UC1Mt. The mice injected with OVA alone or with OVA in combination with MOPC 21 showed similar anti-OVA responses and developed significantly less circulating anti-OVA antibody than mice injected with OVA combined with UC1Mt. The kinetics of the response to OVA was similar in all three groups, and the level of anti-OVA IgG was highest at day 25 in all three experimental groups.

Mice immunized with an isotype matched antibody control did not statistically differ in their anti-OVA IgG response when compared to the control group (p<0.05). The results presented here are representative of three independent experiments.

### Example 2

### UC1MT Alters Anti-OVA Specific Isotype Levels

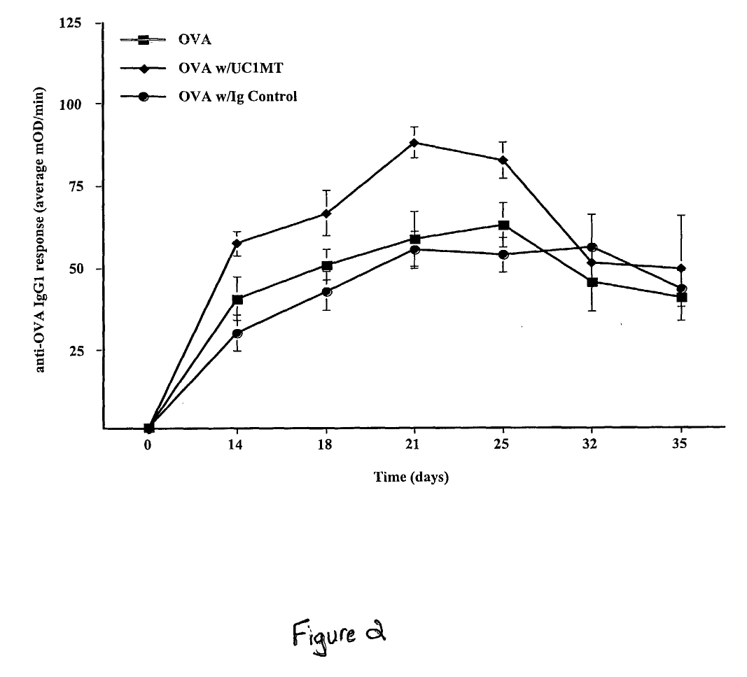

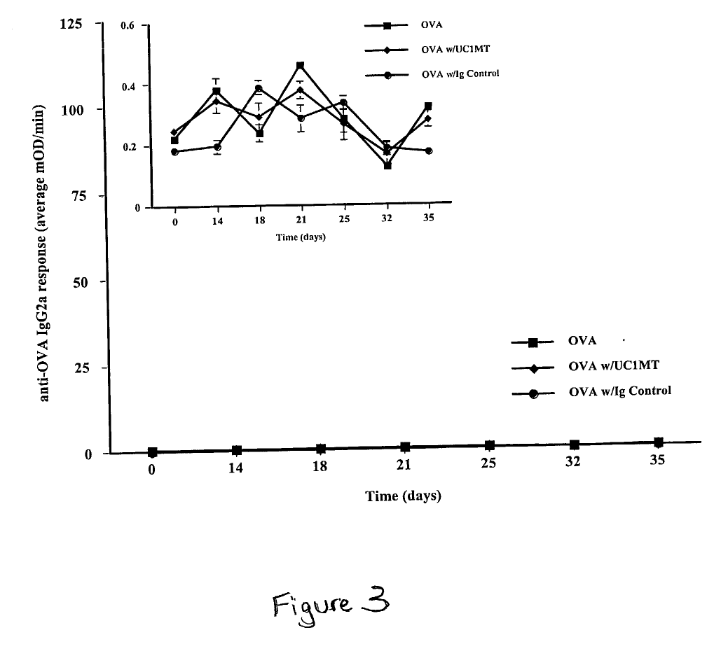

For each of the experiments described in FIG. 1, the anti-OVA specific isotypes of the anti-OVA response were determined. The predominant anti-OVA IgG response enhanced by UC1MT was the IgG₁ response (FIG. 2). In contrast, the IgG₂ₐ mediated anti-OVA response was almost undetectable, and was not affected by co-injection with UC1MT (FIG. 3). Although UC1MT elicited a significant change in the humoral response to OVA, there was no effect of this treatment on total serum Ig levels. Total serum IgG and IgM levels did not significantly differ between any of the experimental groups over the course of this experiment (data not shown). The results show that OVA+UC1MT injected group had significantly increased levels of IgG, when compared to control groups.

### Example 3

### UC1MT Enhances B Cell Differentiation

In order to characterize the activity of antibody producing cells, the number of spot forming cells by ELISPOT assay at day 15 (when anti-OVA levels are significantly different between UC1MT- and MOPC 21-treated animals) were measured. The ELISPOT (Enzyme-Linked Immunospot) assay can be used to determine the number of antibody-secreting cells in a given number of splenocytes (Czerkinsky et al., J. Immunol. Meth. 65:109-21 (1983); Moller and Borrebaeck, J. Immunol. Meth. 79:183-204 (1985)). Briefly, sterile 96-well filtration plates with surfactant-free mixed cellulose ester membrane (Millipore Corp., Bedford, Mass.) were coated with ovalbumin (100 μg/ml) in coating buffer. Control wells were incubated with 100 μg/ml non-fat dry milk. The plates were incubated overnight at 4° C. and washed the following day with PBS/azide using an automated plate washer. Non-specific binding was blocked with 1% Teleostean gelatin in PBS. Single cell suspensions from spleens of immunized mice were prepared in complete RPMI 1640 (starting at 1×10⁶ cells/well) and were added to appropriate wells. After overnight incubation at 37° C. in humidified 5% CO₂ incubator, cells were removed from the plate, and wells were washed three times with PBS with azide. The plates were then incubated with secondary antibody (goat anti-mouse IgG conjugated to alkaline phosphatase) for 2 hours at room temperature and washed again with PBS with azide. Spot forming cells were detected by the addition of 100 μl of BCIP/NBT substrate (Kirkegaard and Perry Laboratories, Gaithersburg, Md.) to each well at room temperature. After the formation of visible spots, the reaction was stopped by washing the plate several times with double distilled H₂O, and spots were counted under the microscope after coding the wells.

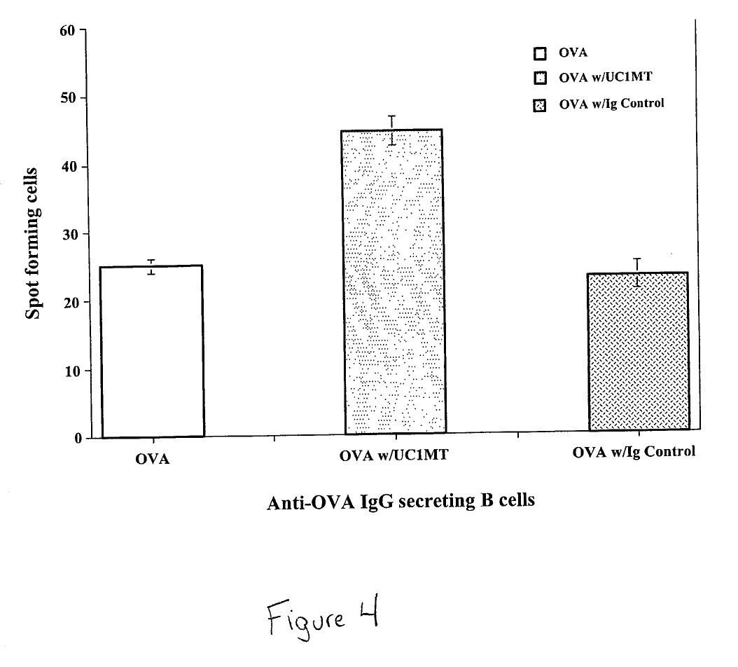

Enumeration of anti-OVA specific IgG plasma cells shows that UC1MT treatment increases numbers of plasma cells when compared to control groups (FIG. 4). No difference in the range of spot sizes in the assay distinguished any group. The results showed that OVA+UC1MT injected group produced significantly more spots when compared to OVA immunized control group (p<0.01).

### Example 4

### Expression of Cell Surface Markers

The relative proportion of B cell and T cell subsets in the spleens from each experimental group were determined by flow cytometry. To determine the expression of cell surface antigens, splenocytes (1×10⁶ cells/well) depleted of erythrocytes by hypotonic lysis were obtained from immunized mice and were pre-incubated with 10% goat serum (GIBCO) in PBS for 1 hour. After this incubation, cells were washed twice with FACS buffer (PBS, 5% FBS, 0.1% NaN₃) and incubated with conjugated anti-mouse IgG-FITC (immunoPure, PIERCE-Rockford, Ill.), anti-mouse IgG+IgM-FITC (Tago Inc., Burlingame, Calif.), anti-mouse CD4-FITC (PharMingen, Becton Dickinson Co., San Diego, Calif.) or anti-mouse CD8-APC (PharMingen) for 30 minutes. After another series of washes, analysis was performed with a Becton Dickinson FACSCalibur (Mountain View, Calif.) using Cellquest 3.2.1 application software.

Spleens were harvested from mice treated as described in FIG. 1 at day 15 of the experiment, where antibody responses had been found to be different in the OVA+UC1MT group. As shown in FIG. 5, the relative frequencies of T and B cells in the spleen of animals from each group were not significantly different, nor were there any differences in the major T cell subpopulations in each group. These results are representative of two independent experiments.

### Example 5

### Binding of Metallothionein to Splenocyte Cell Surfaces

In light of previous experiments that showed that biotinylated metallothionein could bind to the splenocyte plasma membrane (Borghesi et al., Toxicology 108:129-40 (1996)), the kinetics of this interaction using cells cultured with or without 20 μM metallothionein for 15 or 40 hours were examined. Splenocytes from BALB/cByJ mice were incubated in mixed gas (10% CO₂, 7% O₂, 83% N₂) in the absence or presence of metallothionein (20 μM/well) for 15 or 40 hours. After incubation, splenocytes were counted and diluted in FACS buffer (PBS, 5% FBS, and 0.1% NaN₃). For flow cytometry analysis, non-specific binding to splenocytes (1×10⁶ cells/well) was blocked by incubation with rabbit IgG (Sigma) in PBS on ice for 45 minutes. Then, cells were washed with FACS buffer and incubated with appropriate dilutions of UC1MT-FITC or MOPC 21-FITC on ice for 40 minutes. After washing the cells three times with FACS buffer, analysis was performed with a FACSCalibur. In addition, splenocytes from C57BL/6J-Hcph^{mev} mev/mev and +/mev mice were used in some experiments. C57BL/6J-Hcph^{mev} mev/mev mice have been reported to have elevated levels of serum metallothionein (Lynes et al., Metallothionein IV, Klassen. Basel, Birkhauser, pp. 437-44 (1999)). Splenocytes from both types of mice were incubated with UC1MT-FITC in the absence of exogenous metallothionein to determine the presence of metallothionein on the surface of naive autoimmune splenocytes. The Student's t-test was used to determine significant differences between control and treated group of mice. Differences were considered statistically significant when p<0.05.

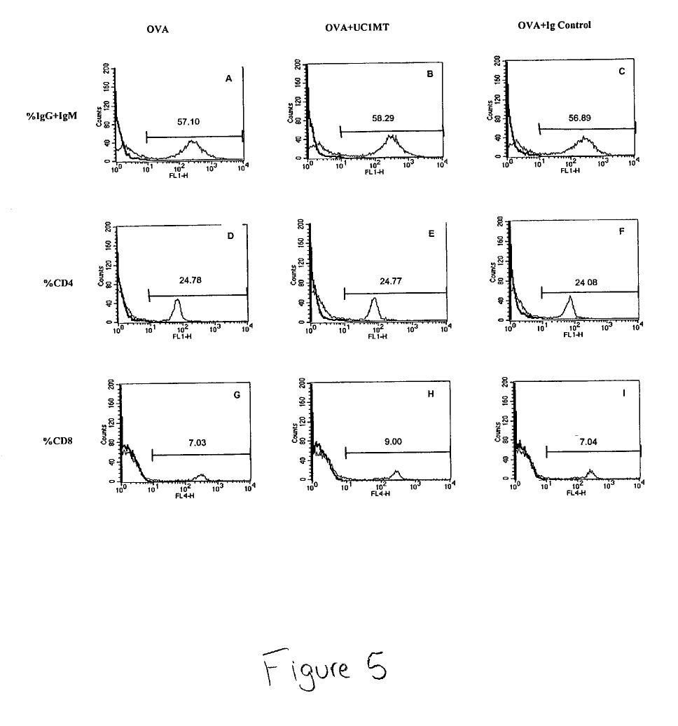

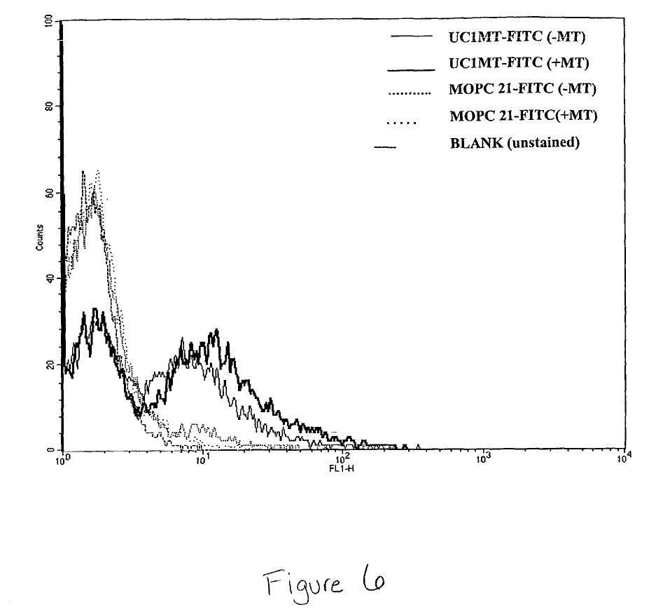

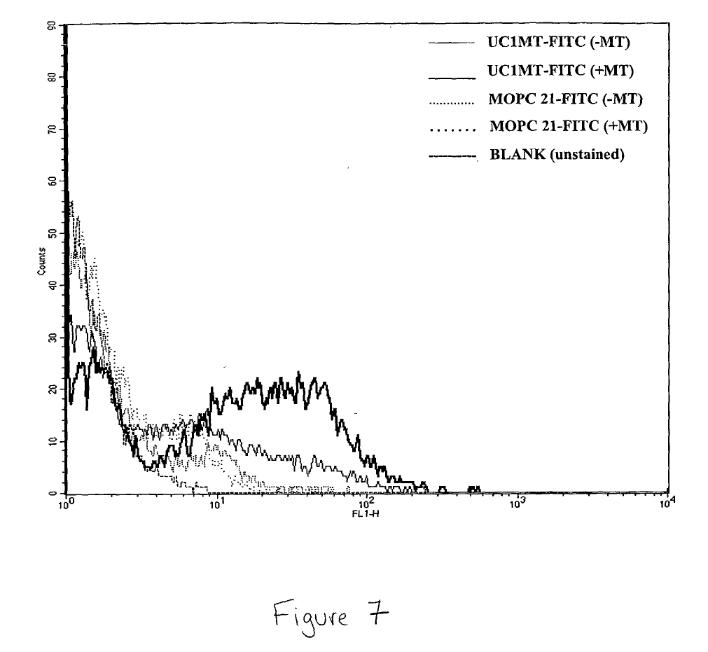

Following culture, the cells were washed and then incubated with UC1MT-FITC or MOPC 21-FITC. After incubation with metallothionein for 15 hours in culture, cells were labeled with UC1MT-FITC at higher levels than those cells incubated with the isotype control (FIG. 6). While incubation with metallothionein produced cells that could be detected with UC1MT-FITC, cells incubated for 15 hours in the absence of exogenous metallothionein could also be detected with this antibody (albeit in lower numbers and at a slightly lower mean fluorescent intensity). A similar phenomenon was observed in 40 hour cultures: the cells incubated in the presence of metallothionein labeled most intensely with UC1MT-FITC, but an appreciable number of cells incubated in the absence of metallothionein were also labeled with UC1MT-FITC (FIG. 7). Lymphocytes harvested directly from naive normal animals were not labeled with UC1MT-FITC (see FIG. 8).

### Example 6

### Binding of Metallothionein to Surfaces of Splenocytes in Autoimmune Mice

The presence of metallothionein on the surfaces of splenocytes obtained from C57BL/6J-Hcph^{mev} mev/mev autoimmune mice was investigated. In previous work it was shown that there is a significant amount of circulating metallothionein in the serum from these mice (Lynes et al., Metallothionein IV, Klassen. Basel, Birkhauser, pp. 437-44 (1999)). Splenocytes from mev/mev homozygotes, which experience a severe form of autoimmunity, were significantly stained with the UC1MT-FITC antibody, while splenocytes from heterozygous littermate controls which do not display this severe autoimmune disease remained unstained (FIG. 8).

### Example 7

### Effects of UC1MT of the Efficacy of a Peptide Vaccine for FMD

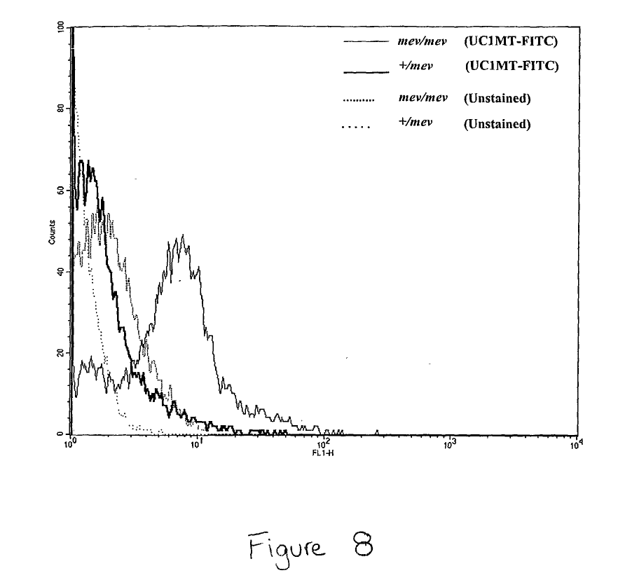

A polypeptide-based vaccine has been developed to induce systemic antibodies to FMD. The polypeptides are based of the G-H loop region of the FMD VP1 structural protein (FIG. 9). Immunization of guinea pigs with the FMDV (VP1-A12₁₄₁₋₁₅₉) polypeptide fragment in the presence of the UC1MT antibody was performed. Guinea pigs were immunized with 100 micrograms of FDV polypeptide in the presence of RIBI adjuvant according to the adjuvant manufacturer's instructions (Sigma Chemical Co., St. Louis, Mo.). RIBI is an adjuvant system based on purified microbial components. Animals were immunized sub-cutaneously, (200 ul at each of two sites) and intraperitoneally on day 0 and day 10. Blood samples were collected by cardiac puncture of anesthetized animals on the days indicated.

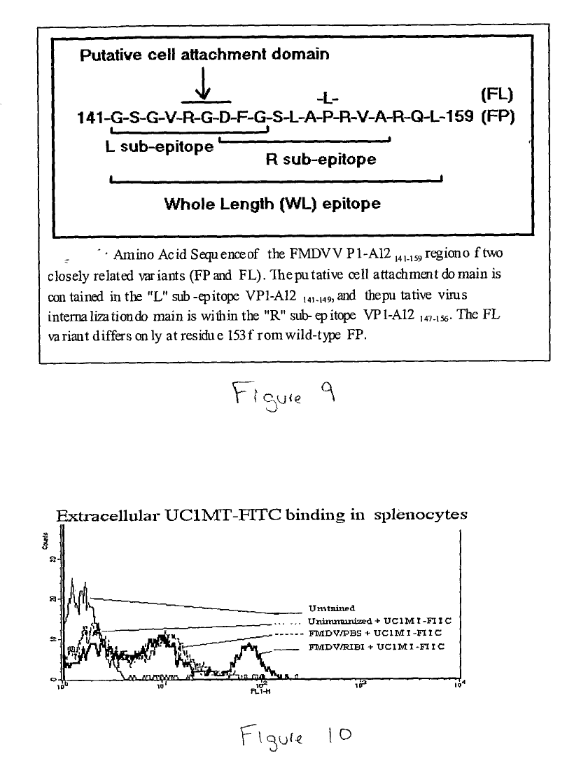

FIGS. 10 and 11 show FACS data for guinea pigs immunized with the FMD peptide, PBS buffer and UC1MT, the FMD peptide, RIBI adjuvant and UC1MT, and no peptide with UC1MT. Metallothionein is found on leukocytes and splenocytes when animals have been immunized with peptide in the presence of RIBI adjuvant and UC1MT, but not when animals are unimmunized or immunized without the RIBI adjuvant. These results show that the UC1MT monoclonal antibody recognizes guinea pig metallothionein. These results also show that use of the RIBI adjuvant increases the amount of surface bound metallothionein.

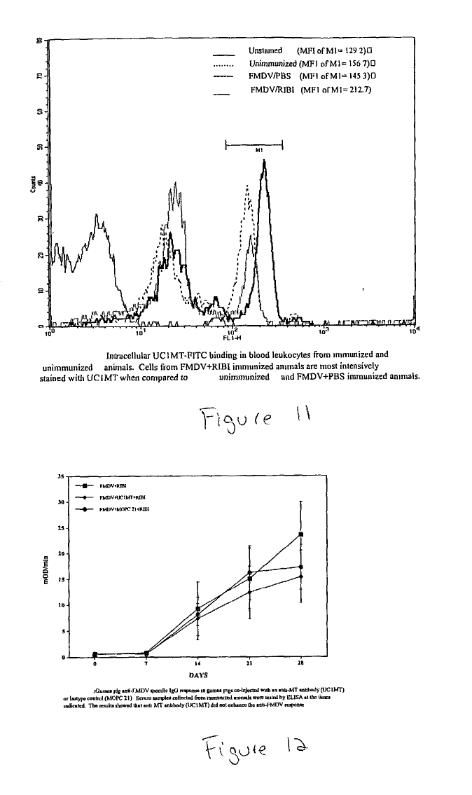

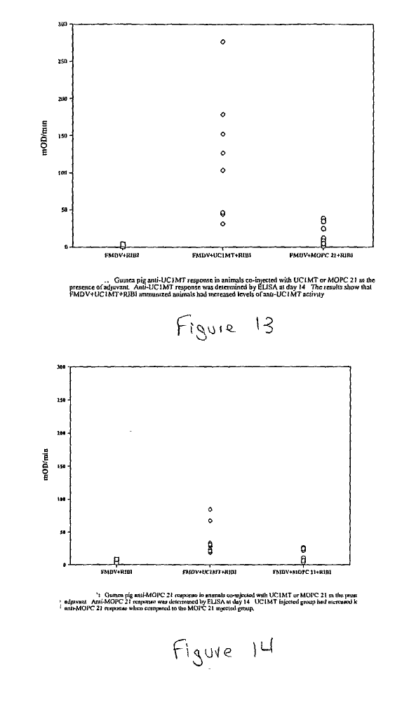

The optimized conditions from the FACS experiments were used to evaluate the capacity of UC1MT to enhance the anti-FMD response upon vaccination with the FMD peptides (FMDV). Three groups of age and sex-matched animals were immunized with FMDV in the presence of RIBI adjuvant. The first group also received vehicle, the second was also injected with UC1MT, and the third was injected with the isotype control MOPC 21 IgG1,κ. As in the mouse studies, it was predicted that the guinea pigs that were immunized in the presence of UC1MT would show an enhanced humoral response to FMDV. Unfortunately, this did not occur (FIG. 12). There was no effect on the anti-FMDV antibody level, nor on the composition of the blood leukocyte populations (data not shown). One possible explanation for this outcome was that the guinea pigs produced a rapid anti-mouse Ig antibody response, so we tested for anti-mouse activity in serum harvested from each guinea pig at day 14 after the start of the experiment (following injections at day 0 and day 10). FIG. 13 shows that there was substantial anti-UC1MT activity produced in animals injected with UC1MT. This appears to be an anti-idiotypic antibody since the animals injected with MOPC 21 do not produce reactivity against the UC1MT antibody (the UC1MT and MOPC 21 antibodies are both produced by BALB/c cells, so there cannot be any allotypic determinants that distinguish these antibodies). Moreover, when these sera were tested against the MOPC 21 in an ELISA, there was far less anti-mouse IgG antibody produced (FIG. 4). We hypothesize that there is something unusual about the UC1MT antibody. It may be that the presence of metallothionein in the context of normal immune responses provokes a low level anti-metallothionein and related anti-idiotypic response, and that injection of UC1MT enlarges that response. If so, this would suggest that both metallothionein and anti-metallothionein are intrinsic regulators of immune function.

All references cited herein are incorporated by reference, as well as U.S. Pat. Nos. 6,168,923; 6,090,406; and 5,928,644.

While preferred embodiments have been shown and described, various modifications and substitutions may be made thereto without departing from the spirit and scope of the disclosure. Accordingly, it is to be understood that the present disclosure has been described by way of illustration and not limitation.

What is claimed is:

1. A method of treating a subject comprising administering to the subject a composition comprising an amount of an anti-metallothionein antibody effective to stimulate a humoral immune response in the subject.

2. The method of claim 1 wherein the anti-metallothionein antibody is UC1Mt.

3. The method of claim 1 wherein the subject is a mammal.

4. The method of claim 3 wherein the subject is a human.

5. The method of claim 4 wherein the human has been treated with a treatment selected from the group consisting of irradiation, chemotherapy, and a combination of the foregoing treatments.

6. The method of claim 4 wherein the human has an autoimmune disease.

7. The method of claim 4 wherein the human has been exposed to an immunosuppressive environmental toxicant.

8. The method of claim 4 wherein the human is a neonate.

9. The method of claim 4 wherein the human has had a transplant.

10. The method of claim 1 wherein the anti-metallothionein antibody is a biologically active fragment.

11. The method of claim 10 wherein the biologically active fragment is an Fab fragment.

12. The method of claim 10 wherein the biologically active fragment is an F(ab′)₂ fragment.

13. The method of claim 1 wherein the composition further comprises a compound selected from the group consisting of a carrier, a diluent, an excipient, and combinations comprising one or more of the foregoing compounds.

14. The method of claim 1 wherein the composition further comprises an adjuvant selected from the group consisting of liposomes, cytokines, interleukins, immunostimulatory nucleic acid molecules, immunoadjuvant oils, polycationic polyelectrolytes, and mixtures comprising one or more of the foregoing adjuvants.

15. A method of treating a subject comprising:
administering to the subject an antigen, and administering to the subject a first amount of an anti-metallothionein antibody effective to stimulate a humoral immune response in the subject.

16. The method of claim 15 wherein the anti-metallothionein antibody is UC1MT.

17. The method of claim 15 wherein administering the antigen and administering the anti-metallothionein antibody are simultaneous.

18. The method of claim 15 further comprising administering to the subject a subsequent second amount of an anti-metallothionein antibody effective to stimulate the humoral immune response in the subject

19. The method of claim 15 wherein administering the anti-metallothionein antibody is subsequent to administering the antigen.

20. A method of immunizing a subject comprising administering to the subject a composition comprising a vaccine and an anti-metallothionein antibody.

21. The method of claim 20 wherein the anti-metallothionein antibody is UC1MT.

22. The method of claim 20 wherein the vaccine is a foot and mouth disease vaccine.

23. The method of claim 20 wherein the subject is an agricultural animal.

24. The method of claim 23 wherein the subject is selected from the group consisting of cattle, sheep and swine.

25. A method of staining a cell to detect metallothionein, comprising
contacting the cell with an anti-metallothionein antibody; and
detecting any metallothionein-anti-metallothionein antibody complexes formed.

26. The method of claim 25 wherein the anti-metallothionein antibody is UC1MT.

27. The method of claim 25 wherein the cell is a splenocyte.

28. The method of claim 25 wherein the anti-metallothionein antibody is labeled with fluorescein isothiocyanate.

29. A method of recovering metallothionein from a mixture comprising
contacting the mixture with an anti-metallothionein antibody; and
isolating metallothionein-anti-metallothionein antibody complexes formed.

30. The method of claim 29 wherein the anti-metallothionein antibody is UC1MT.

31. A method of screening for compounds useful for stimulating a humoral immune response, comprising:
contacting a test compound with metallothionein, and
detecting the presence or absence of binding of the test compound to metallothionein;
wherein the presence of binding to metallothionein indicates that the test compound potentially increases the humoral immune response.

32. The method of claim 31 wherein the test compound is a member of a library.

33. The method of claim 31 wherein the test compound interferes with an interaction of metallothionein and a metallothionein binding compound

34. A method to assess the stimulatory activity of a test substance on a humoral immune response, comprising:
contacting metallothionein with the test substance;
detecting the humoral immune response in the presence of the test substance; and
comparing the humoral immune response in the presence of the test substance with the humoral response in the absence of the test substance.

35. A composition comprising an anti-metallothionein antibody and a vaccine.

36. The composition of claim 35, wherein the vaccine is an FMD vaccine.
